(19) United States
(12) Patent Application Publication
(10) Pub. No.: US 2004/0242508 A1
(43) Pub. Date: Dec. 2, 2004

(54) Novel hydroxyalkyl indolocarbazole derivatives, preparation method and pharmaceutical compositions containing the same

(76) Inventors: Michelle Prudhomme, Clermont-Ferrand (FR); Christelle Marminon, Maisons-Laffitte (FR); Pascale Moreau, Cournon D' Auvergne (FR); John Hickman, Paris (FR); Alain Pierre, Les Alluets Le Roi (FR); Bruno Pfeiffer, Saint Leu La Foret (FR); Pierre Renard, Le Chesnay (FR); Jean-Guy Bizot-Espiard, Paris (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/492,876

(22) PCT Filed: Oct. 21, 2002
(86) PCT No.: PCT/FR02/03592

(30) Foreign Application Priority Data
Oct. 22, 2001 (FR) 01/13576

(51) Int. Cl. A61K 31/7056; C07H 19/22; A61K 31/407

(52) U.S. Cl. 514/43; 514/410; 536/27.1; 548/417

(57) ABSTRACT

Compound of formula (I):

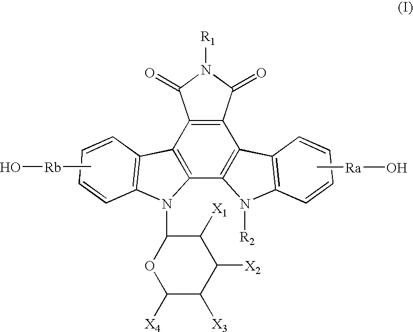

wherein:

R₁ and R₂ each represents a group selected from hydrogen, alkyl, arylalkyl, hydroxy, hydroxyalkyl, dihydroxyalkyl, alkoxy, alkoxyalkyl, amino and aminoalkyl (optionally substituted),

Ra and Rb each represents an alkylene chain,

X₁, X₂ and X₃ each represents a group selected from hydroxy, alkoxy, aryloxy, arylalkoxy, alkyl, amino (optionally substituted), halogen, alkylcarbonyloxy and azido,

X₄ represents a methylidene group or a group of formula —Rc—X₁ as defined in the description,

their isomers and also their addition salts with a pharmaceutically acceptable acid or base.

The present invention relates to new hydroxyalkylindolocarbazole compounds, to a process for their preparation and to pharmaceutical compositions containing them.

The compounds of the present invention are derivatives of rebeccamycin, which has an inhibitory activity in respect of topoisomerase I, rendering it especially useful in the treatment of tumours. Numerous chemical modifications to rebeccamycin have been carried out both in respect of the functional groups present on the molecule (WO 98/07433) and in respect of the positions thereof on the hexacyclic skeleton (WO 00/64917), with the aim of improving the therapeutic properties.

The compounds described by the Applicant surprisingly have a selective inhibitory activity in respect of a family of kinases, and more especially in respect of the kinase GSK-3 (glycogen synthase kinase).

Glycogen Synthase Kinase 3 is present in most human tissue (muscle, liver, pancreas, heart, intestine, . . . ). The enzyme is implicated in the insulin signaling pathway. Thus insulin, by way of the PI₃-kinase pathway, inhibits GSK-3, leading to an increase in the synthesis of reserves in the form of glycogen. GSK-3 also phosphorylates the substrate proteins of insulin, causing a desensitisation of insulin stimulating pathways. Experiments carried out in the Zucker rat (obese and diabetic) have demonstrated that the inhibition of GSK-3 results in stimulation of glucose transport. It was also confirmed that GSK-3 activity was increased in some models or in some pathological situations in animals and in man (type II diabetes). In addition, some elements allowed demonstration that the inhibition of GSK-3 activity enables the prevention of neurone death in subjects affected by neurodegenerative pathologies, and the prevention of the death of healthy cells in a subject suffering from tumour disease and treated with cytotoxic agents.

Compounds capable of inhibiting the synthesis of GSK-3 are thus especially useful for the treatment of type II diabetes, obesity, pathologies of the central nervous system, Alzheimer's disease and Parkinson's disease, and for preventing the apoptosis of normal cells induced by anti-cancer medicanents.

Thus, the compounds described by the Applicant, in addition to being new, unexpectedly exhibit a selective inhibitory activity in respect of glycogen synthase kinase 3, rendering them especially beneficial for use as a medicament in the treatment of the pathologies mentioned above.

The present invention relates more especially to compounds of formula (I):

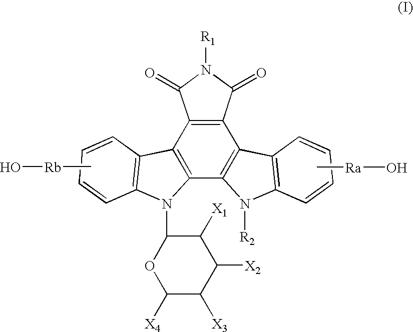

wherein:

R₁ and R₂, which may be identical or different, each represents, independently of the other, a group selected from hydrogen, linear or branched (C₁-C₆)alkyl, aryl-(C₁-C₆)alkyl in which the alkyl moiety may be linear or branched, hydroxy, linear or branched (C₁-C₆)hydroxyalkyl, linear or branched dihydroxy(C₁-C₆)alkyl, linear or branched (C₁-C₆)alkoxy, linear or branched (C₁-C₆)alkoxy(C₁-C₆)alkyl, amino and linear or branched (C₁-C₆)aminoalkyl, the amino moiety in each group being optionally substituted by one or two identical or different groups selected from linear or branched (C₁-C₆)alkyl, aryl and aryl-(C₁-C₆)alkyl in which the alkyl moiety may be linear or branched,

Ra and Rb, which may be identical or different, each represents, independently of the other, a linear or branched (C₁-C₆)alkylene chain,

X₁, X₂ and X₃, which may be identical or different, each represents, independently of the others, a group selected from hydroxy, linear or branched (C₁-C₆)alkoxy, aryloxy, aryl-(C₁-C₆)alkoxy in which the alkoxy moiety may be linear or branched, linear or branched (C₁-C₆)alkyl, amino (optionally substituted by one or two identical or different linear or branched (C₁-C₆)alkyl groups), halogen, linear or branched (C₁-C₆)alkylcarbonyloxy and azido,

X₄ represents a methylidene group or a group of formula —Rc—X₁ wherein Rc represents a single bond or a methylene group and X₁ is as defined hereinabove,

to their isomers and to their addition salts thereof with a pharmaceutically acceptable acid or base,

there being understood by “aryl group” a phenyl or naphthyl group and by “isomers” the optical isomers (racemates, enantiomers and diastereoisomers).

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, etc.

Among the pharmaceutically acceptable bases there may be mentioned, by way of illustration, sodium hydroxide, potassium hydroxide, triethylamine, tert-butylamine, etc..

Preferred R₁ groups of the compounds of the invention are the hydrogen atom, linear or branched (C₁-C₆)alkyl and linear or branched (C₁-C₆)hydroxyalkyl.

The preferred R₂ group of the compounds of the invention is the hydrogen atom.

According to an advantageous variant of the invention, preferred compounds are those of formula (I) wherein Ra and Rb are identical and represent a linear (C₁-C₃)alkylene chain.

Preferred X₁, X₂ and X₃ groups of the compounds of the invention are selected from the groups hydroxy, linear or branched (C₁-C₆)alkoxy and linear or branched (C₁-C₆)alkyl-carbonyloxy.

Preferred X₄ groups of the compounds of the invention are selected from —Rc—X₁ groups wherein Rc represents a methylene group and X₁ represents a group selected from hydroxy, halogen, linear or branched (C₁-C₆)alkoxy, and (C₁-C₆)alkylcarbonyloxy.

According to an advantageous variant of the invention, preferred compounds are compounds of formula (IA):

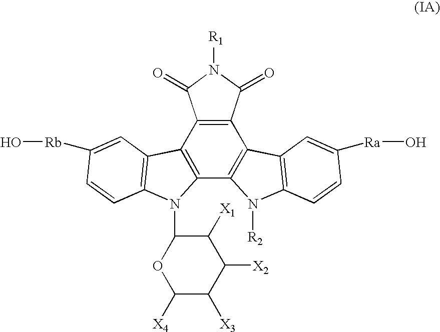

wherein R₁, R₂, Ra, Rb, X₁, X₂, X₃ and X₄ are as defined for formula (I).

The preferred compound of the invention is 3,9-bis(hydroxymethyl)-12-(4-O-methyl-β-D-glucopyranosyl)-12,13-dihydro-5H-indolo[2,3-a]pyrrolo[3,4-c]carbazole-5,7(6H)-dione.

The isomers and the addition salts with a pharmaceutically acceptable acid or base of the preferred compounds form an integral part of the invention.

The present invention relates also to a process for the preparation of compounds of formula (I), which is characterised in that there is used as starting material a compound of formula (II):

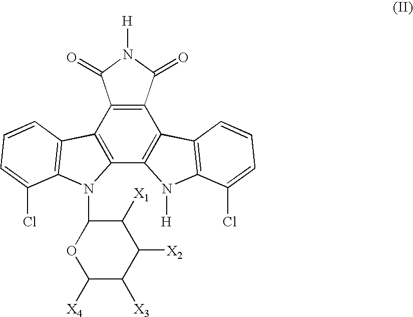

wherein X₁, X₂, X₃ and X₄ are as defined for formula (I),

which compound of formula (II) is subjected to hydrogenolysis conditions in the presence of Raney nickel and sodium hydroxide solution to yield a compound of formula (III):

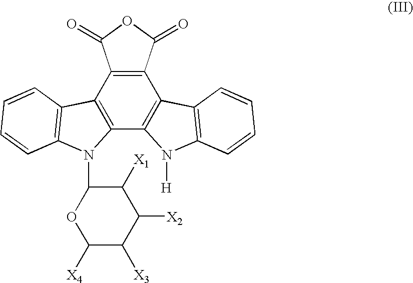

wherein X₁, X₂, X₃ and X₄ are as defined hereinabove,

which compound of formula (III) is subjected to the action of a compound of formula (IV):

R₁—NH₂ (IV)

wherein R₁ is as defined for formula (I),

to yield a compound of formula (V):

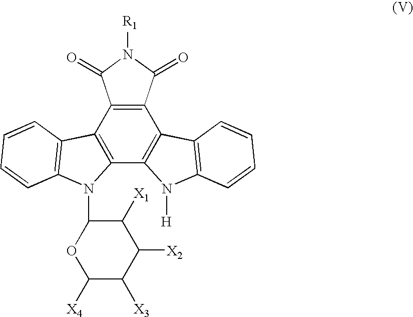

wherein R₁, X₁, X₂, X₃ and X₄ are as defined hereinabove,

which compound of formula (V) is reacted with α,α-dichloromethyl methyl ether in the presence of a Lewis acid to yield a compound of formula (VI):

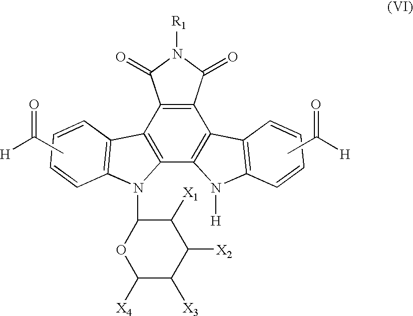

wherein R₁, X₁, X₂, X₃ and X₄ are as defined hereinabove,

the aldehyde functions of which compound of formula (VI) are reduced, by the action of a reducing agent commonly used in organic synthesis, to yield a compound of formula (I/a), a particular case of the compounds of formula (I):

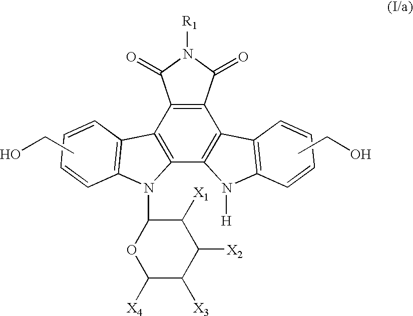

wherein R₁, X₁, X₂, X₃ and X₄ are as defined hereinabove,

which compound of formula (I/a) is converted into its corresponding dihalogenated compound according to customary conditions of organic chemistry, and is then reacted with an alkali cyanide in the presence of dimethyl sulphoxide to yield a compound of formula (VII):

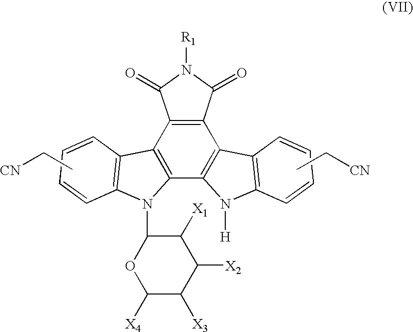

wherein R₁, X₁, X₂, X₃ and X₄ are as defined hereinabove,

which compound of formula (VII) is converted to an ester according to conventional conditions and then subjected to the action of a reducing agent to yield a compound of formula (I/b), a particular case of the compounds of formula (I):

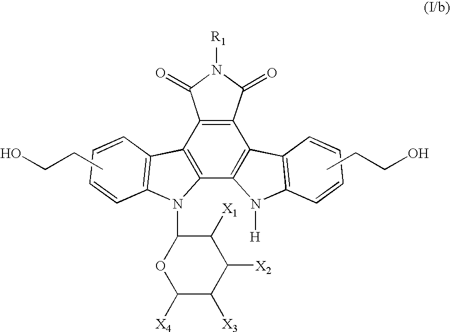

wherein R₁, X₁, X₂, X₃ and X₄ are as defined hereinabove,

which compound of formula (I/b) may be subjected again, and repetitively, to the same series of reactions that resulted in the compounds of formulae (VII) and (I/b) starting from compounds of formula (I/a), to yield a compound of formula (I/c), a particular case of the compounds of formula (I):

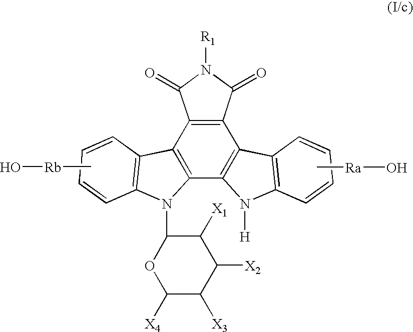

wherein R₁, X₁, X₂, X₃ and X₄ are as defined hereinabove, and Ra and Rb are as defined for formula (I),

which compound of formula (I/c) may be subjected to the action of a compound of formula (VIII):

R₂ₐ—Hal (VIII)

wherein R₂ₐ has the same definition as R₂ in formula (I) with the exception of the definition hydrogen atom, to yield a compound of formula (I/d), a particular case of the compounds of formula (I):

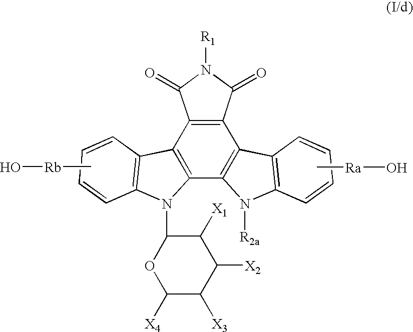

wherein R₁, Ra, Rb, X₁, X₂, X₃, X₄ and R₂ₐ are as defined hereinabove,

the compounds of formulae (I/a) to (I/d) constituting the totality of the compounds of formula (I), which compounds are optionally purified according to conventional purification techniques, may, if desired, be separated into their different isomers according to a conventional separation technique, the substituents X₁, X₂, X₃ and X₄ of which may be modified according to conventional methods of organic synthesis used in the field of sugar chemistry, and which, if desired, are converted into addition salts with a pharmaceutically acceptable acid or base.

The compounds of formulae (II), (IV) and (VIII) are either commercial compounds or are compounds obtained according to conventional methods of organic synthesis readily accessible to the person skilled in the art.

The compounds of formula (I) exhibit a selective GSK-3 (glycogen synthase kinase-3)-inhibiting activity which is altogether surprising. That characteristic property allows them to be used in the treatment of type II diabetes, obesity, pathologies of the central nervous system, Alzheimer's disease, Parkinson's disease and for apoptosis.

The present invention relates also to pharmaceutical compositions comprising as active ingredient at least one compound of formula (I), an isomer thereof, or an addition salt thereof with a pharmaceutically acceptable acid or base, on its own or in combination with one or more pharmaceutically acceptable, inert, non-toxic excipients or carriers.

Among the pharmaceutical compositions according to the invention there may be mentioned more especially those which are suitable for oral, parenteral (intravenous, intramuscular or sub-cutaneous), per- or trans-cutaneous, intravaginal, rectal, nasal, perlingual, buccal, ocular or respiratory administration.

The pharmaceutical compositions according to the invention for parenteral injections comprise, especially, sterile solutions that are aqueous and non-aqueous, dispersions, suspensions or emulsions and also sterile powders for reconstituting injectable solutions or dispersions.

The pharmaceutical compositions according to the invention for solid oral administration comprise especially tablets or dragées, sublingual tablets, sachets, gelatin capsules, granules, and for liquid oral, nasal, buccal or ocular administration comprise especially emulsions, solutions, suspensions, drops, syrups and aerosols.

The pharmaceutical compositions for rectal or vaginal administration are preferably suppositories, and those for per- or trans-cutaneous administration comprise especially powders, aerosols, creams, ointments, gels and patches.

The pharmaceutical compositions mentioned above illustrate the invention but do not limit it in any way.

Among the pharmaceutically acceptable, inert, non-toxic excipients or carriers there may be mentioned, by way of illustration and without implying any limitation, diluents, solvents, preservatives, wetting agents, emulsifiers, dispersing agents, binders, swelling agents, disintegrators, slow-release agents, lubricants, absorbents, suspending agents, colorants, flavourings, etc . . . .

The useful dosage varies in accordance with the age and weight of the patient, the administration route, the pharmaceutical composition employed, the nature and severity of the disorder, and the administration of any associated treatments. The dosage ranges from 0.5 mg to 500 mg in one or more administrations per day.

The Examples which follow illustrate the invention but do not limit it in any way.

The starting materials employed are either known products or are products prepared according to known procedures. The various preparation steps lead to synthesis intermediates for use in the preparation of the compounds of the invention.

The structures of the compounds described in the Examples and in the preparations were determined according to customary spectrophotometric techniques (infra-red, nuclear magnetic resonance, mass spectrometry, . . . ).

### EXAMPLE 1

### 3,9-bis(hydroxymethyl)-1 2-(4-O-methyl-β-D-glucopyranosyl)-12,13-dihydro-5H-indolo[2,3-a]pyrrolo[3,4-c]carbazole-5,7(6H)-dione

Step A: 12-(2,3,6-tri-O-acetyl-4-O-methyl-β-D-glucopyranosyl)-12,13-dihydro-5H-indolo[2,3-a]pyrrolo[3,4-c]carbazole-5,7(6H)-dione.

At 0° C., 1.37 mmol of acetic anhydride and 3 mmol of pyridine are added in succession to 0.136 mmol of dechlorinated rebeccamycin. After stirring for 19 hours at ambient temperature, the reaction mixture is poured onto ice and extracted with ethyl acetate. The organic phase is washed with an Na₂CO₃, solution and then with a saturated NaCl solution, dried over magnesium sulphate and concentrated. Chromatography of the residue on silica gel (ethyl acetate) allows the expected product to be isolated.

Step B: 3,9-Diformyl-12-(2,3,6-tri-O-acetyl-4-O-methyl-β-D-glucopyranosyl)-12,13-dihydro-5H-indolo[2,3-a]pyrrolo[3,4-c]carbazole-5,7(6H)-dione.

2.4 mmol of α,α-dichloromethyl methyl ether are added to a solution of 0.12 mmol of the compound obtained in Step A in 2 ml of dichloromethane. The mixture is cooled to 0° C. and 2.4 Mmol of a 1M solution of TiCl₄ in dichloromethane are added, and the mixture is then stirred at ambient temperature for 24 hours. After hydrolysis, and extraction with dichloromethane, the organic phase is washed with a saturated NaCl solution, dried over magnesium sulphate and concentrated, allowing the expected product to be obtained.

Step C: 3,9-Diformyl-1 2-(4-O-methyl-β-D-glucopyranosyl)-12,13-dihydro-5H-indolo[2,3-a]pyrrolo[3,4-c]carbazole-5,7(6H)-dione.

The compound obtained in Step B is dissolved in 13 ml of methanol, and then 6 ml of an aqueous 30% NH₄OH solution are added. After stirring for 24 hours at ambient temperature, the reaction mixture is evaporated to dryness. The residue is dissolved in an ethyl acetate/tetrahydrofuran mixture, acidified with a 1N hydrochloric acid solution and then extracted with ethyl acetate. The organic phase is dried over magnesium sulphate and evaporated under reduced pressure. Chromatography of the residue on silica gel (cyclohexane/acetone:20/80) allows the expected product to be isolated. Melting point: >300° C.

Step D: 3,9-bis(hydroxymethyl)-12-(4-O-methyl-β-D-glucopyranosyl)-12,13-dihydro-5H-indolo[2,3-a]pyrrolo[3,4-c]carbazole-5,7(6H)-dione.

20 mg of Raney nickel (1/1 by weight in water) are added to a solution of 0.09 mmol of the compound obtained in Step C in 28 ml of methanol. The mixture is stirred for 3 days at ambient temperature under a hydrogen pressure of 1 bar. After filtration through Celite and washing the solid in succession with methanol, tetrahydrofuran and acetone, the solvents are evaporated off. Chromatography of the residue on silica gel (cyclohexane/acetone:1/1) allows the expected product to be isolated. Melting point: >300° C. Infra-red (KBr):v_{CO}=1720, 1740 cm⁻¹; v_{NH.OH}=3100-3600 cm⁻¹

### EXAMPLE 2

### 3,9-bis(hydroxymethyl)-6-methyl-12-(4-O-methyl-β-D-glucopyranosyl)-12,13-dihydro-5H-indolo[2,3-a]pyrrolo-[3,4-c]carbazole-5,7-dione.

Step A: 12-(4-O-methyl-β-D-glucopyranosyl)-12,13-dihydrofuro[3,4-c]-indolo[2,3-a]carbazole-5,7-dione.

A solution of 0.40 mmol of 12-(4-O-methyl-β-D-glucopyranosyl)-12,13-dihydro-5H-indolo[2,3-a]pyrrolo[3,4-c]carbazole-5,7-dione, 420 mg of sodium hydroxide solution and 70 ml of water is heated at reflux for 3 hours, and then diluted, acidified with an aqueous 1N hydrochloric acid solution and extracted with ethyl acetate. The organic phase is washed, dried, filtered and then concentrated under reduced pressure. Chromatography on silica gel (ethyl acetate/cyclohexane:80/20) allows the expected product to be isolated.

Step B: 6-methyl-12-(4-O-methyl-β-D-glucopyranosyl)-12,13-dihydro-5H-indolo[2,3-a]pyrrolo[3,4-c]carbazole-5,7-dione.

0.12 mmol of the compound obtained in Step A and a 2M solution of methylamine in 14 ml of tetrahydrofuran are stirred at 70° C. for 16 hours. After cooling, the reaction mixture is hydrolysed, bringing about the formation of a precipitate. The latter is purified by chromatography on silica gel (ethyl acetate/cyclohexane:80/20), allowing the expected product to be isolated.

Step C: 3,9-bis(hydroxymethyl)-6-methyl-12-(4-O-methyl-β-D-glucopyranosyl)-12,13-dihydro-5H-indolo[2,3-a]pyrrolo[3,4-c]carbazole-5,7-dione.

The product is obtained according to the procedure in Example 1, Steps A to D, using as substrate the compound obtained in the above Step B.

### EXAMPLE 3

### 6-(2-hydroxyethyl)-3,9-bis(hydroxymethyl)-12-(4-O-methyl-β-D-glucopyranosyl)-12,13-dihydro-5H-indolo[2,3-a]pyrrolo[3,4-c]-carbazole-5,7-dione.

Step A: 6-(2-hydroxyethyl)-12-(4-O-methyl-β-D-glucopyranosyl)-12,13-dihydro-5H-indolo[2,3-a]pyrrolo[3,4-c]carbazole-5,7-dione.

A solution of 0.30 mmol of the compound obtained in Step A of Example 2 and 1.3 ml of ethanolamine is stirred at ambient temperature for 1 hour and then poured onto ice and extracted with ethyl acetate. The organic phase is dried, filtered, and then concentrated under reduced pressure. Chromatography on silica gel (ethyl acetate/cyclohexane) allows the expected product to be isolated.

Step B: 6-(2-hydroxyethyl)-3,9-bis(hydroxymethyl)-12-(4-O-methyl-β-D-gluco-pyranosyl)-12,13-dihydro-5H-indolo[2,3-a]pyrrolo[3,4-c]carbazole-5,7-dione.

The product is obtained according to the procedure in Example 1, Steps A to D, using as substrate the compound obtained in the above Step A.

### EXAMPLE 4

### 6-diethylaminoethyl-3,9-bis(hydroxymethyl)-12-(4-O-methyl-β-D-glucopyranosyl)-12,13-dihydro-5H-indolo[2,3-a]pyrrolo[3,4-c]-carbazole-5,7-dione.

Step A: 6-diethylaminoethyl-12-(4-O-methyl-β-D-glucopyranosyl)-12,13-dihydro-5H-indolo[2,3-a]pyrrolo[3,4-c]carbazole-5,7-dione.

26 μl of N,N-diethylethylenediamine are added dropwise to a solution of 60 mg of the compound obtained in Step A of Example 2 dissolved in 7 ml of dry tetrahydrofuran. The reaction mixture is heated at 65° C. for 4 days with the exclusion of light and then cooled and taken up in a mixture (aqueous 1N hydrochloric acid solution/ethyl acetate).

After extraction with ethyl acetate, the organic phase is dried, filtered, and then concentrated under reduced pressure. Chromatography on silica gel allows the expected product to be isolated.

Step B: 6-diethylaminoethyl-3,9-bis(hydroxymethyl)-12-(4-O-methyl-β-D-glucopyranosyl)-12,13-dihydro-5H-indolo[2,3-a]pyrrolo[3,4-c]carbazole-5,7-dione.

The product is obtained according to the procedure in Example 1, Steps A to D, using as substrate the compound obtained in the above Step A.

### EXAMPLE 5

### 3,9-bis(hydroxymethyl)-12-(2,3,6-tri-O-acetyl-4-O-methyl-β-D-glucopyranosyl)-12,13-dihydro-5H-indolo[2,3-a]pyrrolo[3,4-c]-carbazole-5,7(6H)-dione.

The product is obtained according to the procedure in Step D of Example 1, using as substrate the compound obtained in Step B of Example 1.

### EXAMPLE 6

### 3,9-bis(hydroxymethyl)-12-(6-chloro-6-deoxy-4-O-methyl-β-D-glucopyranosyl)-12,13-dihydro-5H-indolo[2,3-a]pyrrolo[3,4-c]-carbazole-5,7(6H)-dione.

Step A: 12-(6-chloro-6-deoxy-4-O-methyl-β-D-glucopyranosyl)-12,13-dihydro-5H-indolo[2,3-a]pyrrolo[3,4-c]carbazole-5,7(6H)-dione.

4 equivalents of PPh₃ and 2 equivalents of CCl₄ are added to a solution of 0.45 mmol of dechlorinated rebeccamycin in 2 ml of pyridine. After stirring at ambient temperature for 3 hours, the reaction mixture is hydrolysed with an aqueous 1N hydrochloric acid solution and then extracted with ethyl acetate. The organic phase is washed, dried, filtered, and then concentrated under reduced pressure.

Step B: 3,9-bis(hydroxymethyl)-12-(6-chloro-6-deoxy-4-O-methyl-β-D-glucopyranosyl)-12,13-dihydro-5H-indolo[2,3-a]pyrrolo[3,4-c]carbazole-5,7(6H)-dione.

The product is obtained according to the procedure in Example 1, Steps A to D, using as substrate the compound obtained in the above Step A.

### PHARMACOLOGICAL STUDY OF THE COMPOUNDS OF THE INVENTION

### EXAMPLE 7

### Inhibitory Activity in Respect of GSK-3

Experimental Protocol

Glycogen synthase kinase 3 was purified starting from Sf9 cells transfected as described in Eur. J. Biochem., 1992, 305-311. The reaction mixture comprises, in a final volume of 30 μl: 1 mg/ml of BSA, 10 mM DTT, 6.7 μM GS-1 peptide as substrate, 15 μM [γ-³²P]ATP (3000 Ci/mmol, 1 mCi/ml), 10 mM MgCl₂, 1 mM EGTA, 25mM Tris-HCl pH=7.5, 50 μg/ml of heparin, and the inhibitor at a given concentration. After 30 minutes at 30° C., 25 μl of the mixture are deposited on Whatman® P81 phosphocellulose paper filters, which are then washed 5 times with 10 ml of phosphoric acid (10 ml/l). The radioactivity of the filters is then counted in the presence of 1 ml of scintillation liquid. The IC₅₀ values are estimated from dose-response curves.

In this test, the compound of Example 1 has an IC₅₀ of 0.03 μM. It is thus active in respect of GSK-3 and that activity is selective, as proved by the results given in Examples 8 and 9 described below.

### EXAMPLE 8

### Inhibitory Activity in Respect of CDK-1

Experimental Protocol

The enzyme was purified from a starfish (Marthasterias glacialis) oocyte homogenate at M phase as described in Eur. J. Biochem, 1997, 243, 527-536 and J. Biol. Chem.,1999, 274, 11977-11986. The reaction mixture comprises, in a volume of 30 μl: 1 mg/ml of histone H1 as substrate, 15 μM [γ-³²P]ATP (3000 Ci/mmol, 1 mCi/ml), 15 mM MgCl₂, 60 mM β-glycerophosphate, 15 mM p-nitrophenylphosphate, 25 mM MOPS pH=7.2, 5 mM EGTA, 1 mM DTT, 1 mM sodium vanadate, and the inhibitor at a given concentration. After 10 minutes' incubation at 30° C., 25 μl of the reaction mixture are removed and treated as described above in the GSK-3 protocol. The IC₅₀ values are estimated from dose-response curves.

In this test, the compound of Example 1 has an IC₅₀ greater than 5 μM, thus demonstrating its low capacity to inhibit that cyclin-dependent protein kinase.

### EXAMPLE 9

### Inhibitory Activity in Respect of CDK5

Experimental Protocol

CDK5 was expressed in E. coli in the form of a GST (Glutathione-S-transferase) fusion protein and purified on a glutathione-agarose affinity column. CDK5 is then activated by p25 (1/1 mixture), prepared in the same manner. The enzymatic activity of the CDK5/p25 complex is measured as described above for CDK1/cyclin B. The IC₅₀ values are estimated from dose-response curves.

In this test, the compound of Example 1 exhibits an IC₅₀ greater than 5 μM, thus demonstrating its low capacity to inhibit that cyclin-dependent protein kinase.

### EXAMPLE 10

### Pharmaceutical Composition for 1000 Tablets each Containing a Dose of 10 mg

| | |
|---|---|
| compound of Example 1 | 10 g |
| hydroxypropyl methylcellulose | 10 g |
| wheat starch | 15 g |
| lactose | 90 g |
| magnesium stearate | 2 g |

1-11. (CANCELED).

12. A compound selected from those of formula (I): 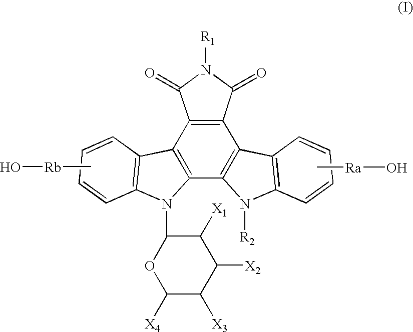
wherein:
R₁ and R₂, which may be identical or different, each represents, independently of the other, a group selected from hydrogen, linear or branched (C₁-C₆)alkyl, aryl-(C₁-C₆)alkyl in which the alkyl moiety may be linear or branched, hydroxy, linear or branched (C₁-C₆)hydroxyalkyl, linear or branched dihydroxy(C₁-C₆)alkyl, linear or branched (C₁-C₆)alkoxy, linear or branched (C₁-C₆)alkoxy(C₁-C₆)alkyl, amino and linear or branched (C₁-C₆)aminoalkyl, the amino moiety in each group being optionally substituted by one or two identical or different groups selected from linear or branched (C₁-C₆)alkyl, aryl and aryl-(C₁-C₆)alkyl in which the alkyl moiety may be linear or branched,
Ra and Rb, which may be identical or different, each represents, independently of the other, a linear or branched (C₁-C₆)alkylene chain,
X₁, X₂ and X₃, which may be identical or different, each represents, independently of the others, a group selected from hydroxy, linear or branched (C₁-C₆)alkoxy, aryloxy, aryl-(C₁-C₆)alkoxy in which the alkoxy moiety may be linear or branched, linear or branched (C₁-C₆)alkyl, amino (optionally substituted by one or two identical or different linear or branched (C₁-C₆)alkyl groups), halogen, linear or branched (C₁-C₆)alkylcarbonyloxy and azido,
X₄ represents a methylidene group or a group of formula —Rc—X₁ wherein Rc represents a single bond or a methylene group and X₁ is as defined hereinabove,
its optical isomers and addition salts thereof with a pharmaceutically acceptable acid or base,
it being understood that aryl may be a phenyl or naphthyl.

13. A compound of claim 12, wherein R₁ represents hydrogen, linear or branched (C₁-C₆) alkyl or linear or branched (C₁-C₆)hydroxyalkyl.

14. A compound of claim 12, wherein R₂ represents hydrogen.

15. A compound of claim 12, wherein Ra and Rb are identical and represent a linear (C₁-C₃) alkylene chain.

16. A compound of claim 12, wherein X₁, X₂ and X₃ each represents a group selected from hydroxy, linear or branched (C₁-C₆)alkoxy and linear or branched (C₁-C₆) alkylcarbonyloxy.

17. A compound of claim 12, wherein X₄ represents a group of formula —Rc—X₁ wherein Rc represents a methylene group and X₁ represents a group selected from hydroxy, halogen, linear or branched (C₁-C₆)alkoxy, and (C₁-C₆)alkylcarbonyloxy.

18. A compound of claim 12, which is a compound of formula (IA): 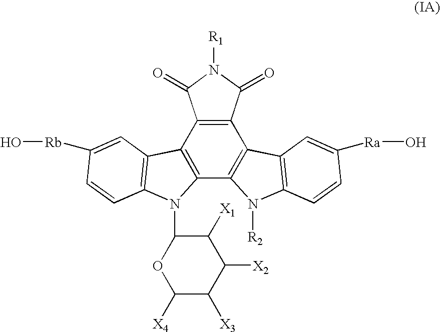
wherein R₁, R₂, Ra, Rb, X₁, X₂, X₃ and X₄ are as defined for formula (I), its optical isomers and additional salts thereof with a pharmaceutically acceptable acid or base.

19. A compound of claim 12 which is 3,9-bis(hydroxymethyl)-12-(4-O-methyl-β-D-glucopyranosyl)-12,13-dihydro-5H-indolo[2,3-a]pyrrolo[3,4-c]carbazole-5,7(6H)-dione.

20. A method for treating a living animal body afflicted with type II diabetes, obesity, pathologies of the central nervous system, Alzheimers disease, Parkinson's disease and conditions treatable by an inhibitor of apoptosis of normal cells caused by anti-cancer treatments, comprising the step of administering to the living animal body an amount of a compound of claim 12 which is effective for alleviation of said conditions.

21. A pharmaceutical composition useful as inhibitor of the glycogen synthase kinase GSK-3, comprising as active principle an effective amount of a compound as claimed in claim 12 together with one or more pharmaceutical acceptable excipients or vehicles.

22. A pharmaceutical composition useful for treating diabetes, obesity, pathologies of the central nervous system, Alzheimers disease, Parkinson's disease and conditions treatable by an inhibitor of apoptosis of normal cells caused by anti-cancer treatments, comprising as active principle an effective amount of a compound as claimed in claim 12 together with one or more pharmaceutical acceptable excipients or vehicles.
